(19) United States
(12) Patent Application Publication
(10) Pub. No.: US 2017/0101374 A1
(43) Pub. Date: Apr. 13, 2017

(54) METHOD FOR REMOVING PROTONATED AMINE COMPOUND FROM QUATERNARY AMINE COMPOUND

(71) Applicant: CoorsTek Fluorochemicals, Inc., Arvada, CO (US)

(72) Inventors: Andrew R. Riscoe, Denver, CO (US); Joseph C. Poshusta, Broomfield, CO (US)

(73) Assignee: CoorsTek Fluorochemicals, Inc., Arvada, CO (US)

(21) Appl. No.: 14/879,015

(22) Filed: Oct. 8, 2015

(51) Int. Cl. C07D 207/06

(52) U.S. Cl. CPC C07D 207/06

(57) ABSTRACT

The present invention provides a method and process for removing at least a portion of a protonated amine compound that is present as an impurity or a contaminant in a quaternary amine compound. The method of invention is particularly useful in producing highly purified ionic liquids that have quaternary amine moiety as the cationic moiety.

### STATEMENT REGARDING FEDERALLY FUNDED RESEARCH

This invention was made with government support under grant number IIP-1152040 awarded by the National Science. The government has certain rights in the invention.

### FIELD OF THE INVENTION

The present invention relates to a method and process for removing at least a portion of a protonated amine compound that is present as an impurity or a contaminant in a quaternary amine compound. In particular, the invention relates to production of ionic liquids that comprise a quaternary amine moiety as the cationic portion. Such ionic liquids of the invention are substantially free of protonated amine compound or the tertiary amine compound that is used to produce the quaternary amine moiety.

### BACKGROUND OF THE INVENTION

Quaternary amine compounds are useful in a wide variety of applications including, but not limited to, production of ionic liquids, surfactants, catalysts, etc. Accordingly, various methods have been developed for synthesizing quaternary amine compounds. One of the methods for synthesizing quaternary amine compounds, such as pyrrolidinium bromide salts, utilizes water as a solvent. Use of water as a solvent has been shown to have numerous advantages. In general, synthesis of quaternary amine compounds involves an alkylation of a tertiary amine with an alkylhalide.

While use of water as a solvent simplifies the process, a side reaction involving hydrolysis of the alkylhalide (i.e., haloalkane) can lead to an undesired byproduct, e.g., a protonated ammonium compound. In some cases, separation of the protonated amine compound from the quaternary amine compound can be difficult. In fact, the quaternary amine compound is often used as a mixture containing the protonated amine compound contaminant. While the presence of a protonated amine compound in the quaternary amine compound is not detrimental in many instances, there are instances where the presence of a protonated amine compound in the quaternary amine compound can have a significantly negative impact on its use.

Therefore, there is a need for a method for efficiently removing a protonated amine compound from a quaternary amine compound.

### SUMMARY OF THE INVENTION

Some aspects of the invention provide a method for removing a protonated amine compound from a quaternary amine compound. Typically, the method includes contacting a quaternary amine compound comprising a protonated amine compound with a base under conditions sufficient to remove said protonated amine compound from said quaternary amine compound. Generally, the pKa of a counter acid of said base is higher than the pKa of said protonated amine compound. This difference in pKa ensures equilibrium of acid-base reaction to favor deprotonation of the protonated amine compound. In some embodiments, a quaternary amine compound that is substantially free of protonated amine compound (and/or the tertiary amine compound) is produced. As used herein, the term “substantially free” refers to quaternary amine compound having about 0.5% or less, typically 0.1% or less, often 0.01% or less, more often 0.005% or less, still more often 0.001% or less, and most often 0.0001% or less of protonated amine compound (and/or the tertiary amine compound).

Still other aspects of the invention provide a process for producing an ionic liquid comprising a quaternary amine cation, said process comprising:
(a) contacting a tertiary amine compound with an alkylating compound to produce a cationic portion of an ionic liquid, wherein said cationic portion of said ionic liquid is a quaternary amine compound;
(b) admixing said quaternary amine compound with an acid or salt of anionic portion of said ionic liquid to produce said ionic liquid; and
(c) contacting said ionic liquid with a base under conditions sufficient to remove at least a portion of a protonated amine compound that is present in said ionic liquid of said step (b).

In some embodiments, the pKa of a counter acid of said base is higher than the pKa of said protonated amine compound. In other embodiments, said ionic liquid comprises about 100 ppm or less of said protonated amine compound. The protonated amine compound can be removed by adsorption or any other methods known to one skilled in the art. At least a portion of the tertiary amine compound can be removed as a vapor, e.g., by distillation, using vacuum, by purging with an inert gas, or a combination thereof.

Yet other aspects of the invention provide a process for producing a quaternary amine compound comprising:
(a) reacting a tertiary amine compound with an alkylating agent in an aqueous solution under conditions sufficient to produce a crude quaternary amine compound;
(b) adding a base to said crude quaternary amine compound under conditions sufficient to deprotonate a protonated tertiary amine compound that may be present in said crude quaternary amine compound to form said tertiary amine compound, wherein the pKa of a counter acid of said base is higher than the pKa of said protonated tertiary amine compound; and
(c) removing said tertiary amine compound produced in said step (b) to produce said quaternary amine compound.

In some embodiments, the crude quaternary amine compound is isolated prior to said step (b) of adding said base. Yet in other embodiments, the process can further include the step of metathesizing said crude quaternary amine compound to produce an ionic liquid form of said crude quaternary amine compound prior to said step (b) of adding said base. Still in other embodiments, the tertiary amine compound is removed from said quaternary amine compound by adsorption, as a vapor, solvent extraction or a combination thereof. Typically, the amount of said tertiary amine compound in said quaternary amine compound is about 100 ppm or less.

### DETAILED DESCRIPTION OF THE INVENTION

While the present invention is applicable for removing a protonated amine compound that is present in any quaternary amine compound, the present invention will now be described with reference to purifying a quaternary amine compound used in ionic liquids, i.e., ionic liquids in which the cationic portion is a quaternary amine moiety. Synthesis of pyrrolidinium bromide salts in water as a solvent has been shown to have numerous advantages.

In general, these reactions involve quaternization of a tertiary amine (an organic base) compound I by alkylating the tertiary amine I with an alkylhalide II as illustrated below:

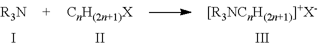

While use of water as a solvent simplifies the reaction and the work-up, a side reaction involving hydrolysis of the alkylhalide II can lead to an undesired byproduct of a protonated tertiary amine compound, i.e., a protonated amine compound.

Without being bound by any theory, it is believed that the reaction involves hydrolysis of the alkylhalide II to form the corresponding alcohol IV and the acid halide V via the following reaction:

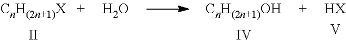

The acid halide V that is produced in the above reaction can neutralize the basic tertiary amine I to form the protonated tertiary ammonium halide salt VI by the following reaction:

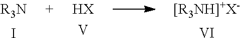

For example, bromopropane hydrolyzes slowly in water to produce HBr and propanol. The HBr generated then reacts with N-methyl pyrrolidine (i.e., mPyr) to form hydrogen methyl pyrrolidinium bromide (“Pyr1H Br”), a protonated amine bromide salt.

The reaction to form a quaternary amine halide salt III is typically the first step in preparing an ionic liquid, where the quaternary amine cation of compound III is used as the cationic portion of the ionic liquid. The desired anion is manufactured in a separate process and is typically provided as an acid or alkali metal salt of the desired anion. If the ionic liquid is hydrophobic, the desired cation and anion can be easily paired by metathesis in water. The undesired cation and anion are highly water soluble and are readily removed in the aqueous phase. The reaction scheme below illustrates a general reaction for producing an ionic liquid by metathesis in water:

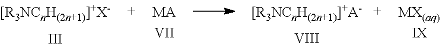

where M is a proton or mono-valent metal cation. If the resulting salt MX (compound IX) is highly water soluble, then an ionic liquid VIII can be isolated at more than 95% purity. Any protonated amine compounds that maybe present will also participate in the metathesis as illustrated by the following reaction scheme:

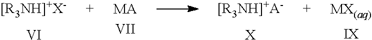

The protic cation metathesis product X may also be hydrophobic and therefore difficult to remove from the desired ionic liquid VIII by extraction with water. For example, in the metathesis of the N-methyl-N-propyl pyrrolidinium bromide (“Pyr13 Br”) with a bis(fluorosulfonyl)imide (“FSI”) salt, the presence of a small amount of N-methyl pyrrolidinium bromide (“Pyr1H Br”)—which may be present in the starting Pyr13 Br—leads to formation of N-methyl pyrrolidinium bis(fluorosulfonyl)imide (“Pyr1H FSI”), which is also an ionic liquid. The presence of this byproduct (Pyr1H FSI) is low in concentration, but its removal is very difficult. For example, attempts to remove Pyr1H FSI from Pyr13 FSI via extraction with water have shown to be inefficient. Attempts to remove Pyr1H FSI by adsorption on carbon have also been shown to be ineffective.

The presence of protonated amine compounds can be detected and quantified by electrochemical methods or by cation chromatography. In addition, on cyclic voltammetry, the integrated area of the current density versus voltage curve for the reduction can be correlated to the concentration of protonated amine compound. The protonated amine compound can be reduced on a platinum electrode during cyclic voltammetry. This electrochemical reducibility of protonated amine compound renders its presence in electrolytes undesirable when used in batteries and capacitors.

Some aspects of the invention provide a method for removing a protonated amine compound from a quaternary amine compound. As used herein, the term “protonated amine compound” refers to an amine compound in which the nitrogen atom of the amine compound is protonated. Typically, the protonated amine compound is a tertiary amine compound that is protonated (i.e., “protonated tertiary amine compound”). However, it should be appreciated that the scope of the invention is not limited to tertiary amine compounds as methods of the invention can also be used to reduce or prevent formation/production of protonated monoalkyl and dialkyl amine compounds during an alkylation reaction in an aqueous solution. The term “amine compound” refers to a compound having a basic nitrogen atom with a lone pair. Amine compounds are derivatives of ammonia, wherein one or more hydrogen atoms have been replaced by a hydrocarbon substituent such as an alkyl or aryl group or any other hydrocarbon moieties known to one skilled in the art. It should be appreciated that two or more hydrocarbon moieties that are attached to the nitrogen atom of the amine compound can form a ring system. “Hydrocarbon” can include one or more unsaturations, e.g., carbon-carbon double bond or triple bond. Hydrocarbon can be a straight chain, branched chain, mono- or bi-cyclic ring system, or a combination thereof. In addition, hydrocarbon can also include other functional groups, such as ethers, hydroxides (protected and/or unprotected), esters, carboxylic acids, sulfates, etc. as long as the functional group does not substantially interfere with alkylation reaction between an alkylating agent and the amine functional group. The nitrogen atom of the amine compound can be a part of a heterocyclic ring system or a heteroaryl ring system. Exemplary heterocyclic ring systems having one or more nitrogen atoms include, but are not limited to, piperidinyl, piperazinyl, morpholinyl and thiomorpholinyl, etc. Exemplary heteroaryl ring systems having one of more nitrogen atoms include, but are not limited to, pyridyl, triazolyl, imidazolyl, isoxazolyl, pyrrolyl, pyrazolyl, pyrimidinyl, benzotriazolyl, indolyl, isoindolyl, benzoxazolyl, benzimidazolyl, etc.

In some embodiments of the invention, the protonated amine compound is an ionic liquid. The term “ionic liquid” refers to a salt in which the ions are poorly coordinated, which results in these compounds being liquid below, for example, 100° C., or even at room temperature (i.e., room temperature ionic liquids or RTIL's) or below. Ambient temperature (i.e., room temperature or about 20° C.) ionic liquids are often used as electrolytes. Many ionic liquids are non-volatile, non-flammable, have a wide electrochemical stability window and high ionic conductivity. The terms “quaternary amine compound” and “quaternary amine cation compound” are used interchangeably herein and refer to a nitrogen atom containing compound in which the nitrogen atom is attached to four hydrocarbon moieties. Thus, as used herein, the term quaternary amine compound explicitly excludes protonated amine compounds. Typically, the quaternary amine compound is derived from alkylation of the unprotonated amine compound.

Methods of the invention remove at least 80%, typically at least 90%, and often at least 99% of the protonated amine compound that is present in the quaternary amine compound. Some aspects of the invention provide a quaternary amine ionic liquid that has a minute amount of protonated tertiary ammonium ionic liquid as an impurity. Such quaternary amine ionic liquids of the invention have less than 100 ppm, typically less than 10 ppm, and often less than 1 ppm protonated tertiary ammonium ionic liquid. Other aspects of the invention provide a quaternary amine ionic liquid that is produced by the method of the invention disclosed herein.

Methods of the invention include contacting the quaternary amine compound comprising a protonated amine compound with a base under conditions sufficient to remove the protonated amine compound from the quaternary amine compound. The pKa of the counter acid of the base used is higher than the pKa of the protonated amine compound. In particular, methods of the invention add a base that is significantly stronger in basicity than the tertiary amine compound (e.g., compound I) that is used in producing the quaternary amine compound III. The addition of this stronger base at sufficiently high concentration converts protonated tertiary ammonium cation (e.g., compound III and/or VI) in the product to the unprotonated tertiary amine compound (e.g., compound I) so that it can be readily removed by subsequent processing. When combined with industrial standard purification methods, the organic base impurity can be removed by several orders of magnitude more than the conventional purification methods.

Typically, the pKa of the counter acid of the base is higher than the pKa of the protonated amine compound by at least 0.1, typically by at least 2, and often by at least 5. For example, pKa of protonated triethylamine or other simple protonated trialkylamine compounds is about 11 and the pKa of the counter acid of hydroxide, i.e., water, is about 16. Thus, when a hydroxide, such as NaOH, KOH, Ca(OH)₂, LiOH, Mg(OH)₂, etc., is added to triethylammonium chloride (Et₃N.HCl), the equilibrium of the reaction is such that substantially all of the triethylammonium chloride is converted to triethylamine. Thus, in general and without being bound to any theory, it is believed that addition of strong base in sufficient amount to the quaternary amine compound having a protonated amine compound impurity or contaminant, in particular quaternary amine compound that is produced by alkylation in an aqueous solution, raises the pH of the system above the point at which the amount of protonated amine cation is reduced to form a neutral tertiary amine by a simple acid-base reaction. The acid-base reaction involving the base and the protonated amine compound is reversible and the equilibrium of the reaction is pH dependent. High pH favors the forward reaction of the following reaction scheme:

[R₃NH]⁺ [symbol] R₃N+H⁺

For many tertiary amines, the equilibrium constant of this reaction is known. For example, N-methyl pyrrolidine (“mPyr”) has a pKb of 3.45 (pKa=10.6). The equilibrium relationship between the protonated form and the deprotonated form is given by the following equation:

Kₐ=([H⁺]×[R₃N])/[R₃NH⁺]

It should be noted that in the above equation, brackets represent concentration of the species in molarity. Rearranging for the concentration of protons yields the following equation:

10^{-pH}=[H⁺]=Ka×([R₃NH⁺]/[R₃N])

It is desired to reduce the ratio of protonated amine compound to amine base, i.e., the unprotonated amine compound. Thus, the pH of the system should be larger than pKₐ of the protonated amine compound in order to have a ratio of protonated amine compound to tertiary amine base that is less than 1. In a system where the pH is substantially less than pKₐ of the protonated amine compound, the protonated amine compound will be stable and remain protonated. If the pH conditions that favor the forward deprotonation of the protonated amine compound are maintained after the alkylation reaction to produce the quaternary amine compound, then the protonated amine compound will be deprotonated and converted back to the tertiary amine compound. For example, at a pH of 11.6 the concentration of protonated amine compound is about 1/10 of the mPyr concentration and at pH of 12.6, the ratio decreases to 1/100.

The pH of the reaction solution can be increased by adding a base substantially stronger than the tertiary amine compound. For example, hydroxides, such as alkali metal hydroxides and alkaline earth metal hydroxides, are strong bases that can raise the pH with a small addition. If some alkyl halide is present in the product solution to be treated, some hydroxide base will be consumed by hydrolysis of alkyl halide to generate water and an alkali metal salt. If the hydrolysis reaction is slow, then the effect of lost base will also be small.

After treating with base, the product solution will contain primarily a desired quaternary amine halide salt and water with smaller amounts of excess alkali metal hydroxide, excess alkyl halide, an alcohol and alkali metal halide salt from hydrolysis of the alkyl halide and the tertiary amine generated from reverting the protonated amine at high pH. When used to make a hydrophobic ionic liquid in a metathesis reaction, traces of these contaminants may remain in the ionic liquid phase. These contaminants can be removed using multiple approaches. In one approach, subsequent extraction of the metathesis product with water will effectively remove the water soluble species (excess alkali metal hydroxide and alkali metal halide salt). In some instances, the excess alkyl halide, corresponding alcohol, and reverted tertiary amine partition into the ionic liquid phase and not be removed as effectively. For relatively short alkyl chains in the alkyl halide, however, these contaminants will have high vapor pressure and can be removed effectively from the ionic liquid by distillation or vapor stripping methods or by adsorption on activated carbon.

In an alternative approach, the aqueous quaternary amine compound solution can be treated with activated carbon after the addition of base to remove reverted protonated amine and any excess alkyl halide. After carbon treating, the metathesis reaction can be carried out and followed with water extractions to remove water soluble contaminants.

The above discussion presents an example mechanism involving alkylhalides as alkylation agents that can also hydrolyze in water to generate acidic species that lead to protonated amine compound, i.e., protonated tertiary amine compound, contamination. Other alkylating agents and hydrolysable species in the reaction can also benefit from the invention in prevention of the formation of protonated amine compounds. For example, without being bound by any theory dialkylsulfates are well known alkylating agents that can also hydrolyze according to the following reaction:

(CₙH₍₂ₙ₊₁₎)₂SO₄+H₂O→CₙH₍ₙ₊₁₎OH+HSO₄CₙH₍₂ₙ₊₁₎

where an alcohol and an acidic alkylbisulfate byproduct is formed. Furthermore, this invention is useful for systems with acidic contaminants or other constituents that generate acidic components.

In some embodiments, the quaternary amine compound is an ionic liquid. In other embodiments, the quaternary amine compound is a liquid at a standard condition. The term “standard condition” refers to 1 atmospheric pressure at 20° C.

In some embodiments, the quaternary compound is used to produce an ionic liquid. In such embodiments, as discussed above, the quaternary compound is subjected to a metathesis reaction with a corresponding anion of the ionic liquid. The quaternary amine compound produced from alkylation is often isolated and purified to produce an ionic liquid. In preparing an ionic liquid, the base can be added to the quaternary amine compound prior to the metathesis reaction to remove the protonated amine compound. Alternatively, or in addition, the base can be added to the quaternary amine compound after the metathesis reaction to remove the protonated amine compound. Regardless of when the base is added, in some instances the quaternary amine compound and the base are admixed in an aqueous solution to remove the protonated amine compound. The addition of base to the quaternary amine compound deprotonates protonated amine compound that may be present as a contaminant or impurity in the quaternary amine compound. Deprotonation of the protonated amine compound converts the protonated amine compound to its corresponding amine compound. Since the amine compound that is produced from deprotonation of the protonated amine compound is non-ionic, it will have a significantly different physical properties including a significantly lower vapor pressure, thereby allowing a much easier separation from the quaternary amine compound.

In some embodiments, the amine compound produced from deprotonation is removed (i.e., separated) from the quaternary amine compound by adsorption. For example, amine compound is readily adsorbed by carbon (e.g., activated charcoal, etc.), whereas quaternary amine compounds are not readily adsorbed by carbon due to its ionic nature. Moreover, since the amine compound has a significantly higher vapor pressure, it can be removed or separated from the quaternary amine compound as a vapor. For example, the amine compound can be distilled (e.g., under vacuum) away from the quaternary amine compound. It can also be removed from quaternary amine compound by gas stripping, e.g., the amine compound is removed from the quaternary amine compound by flowing a stream of gas (e.g., nitrogen, air, helium, etc.). Such a stream of gas can be bubbled through a solution of quaternary amine compound. If the quaternary amine compound is an ionic liquid, the stream of gas can be bubbled directly into the quaternary amine compound. Alternatively, or in addition, the stream of gas can be flowed across the surface of the quaternary amine compound or its solution.

Other aspects of the invention provide a process for producing an ionic liquid comprising a quaternary amine cation, said process comprising:
(a) contacting a tertiary amine compound with an alkylating compound to produce a cationic portion of said ionic liquid, wherein said cationic portion of said ionic liquid is a quaternary amine compound;
(b) admixing said quaternary amine compound with an acid or salt of an anionic portion of said ionic liquid to produce said ionic liquid; and
(c) contacting said ionic liquid with a base under conditions sufficient to remove at least a portion of a protonated amine compound that is present in said ionic liquid of said step (b),
   wherein the pKa of a counter acid of said base is higher than the pKa of said protonated amine compound. As discussed above, an ionic liquid is a salt that is a liquid at temperature below 100° C., and often at room temperature. The ionic liquid of the invention is comprised of a quaternary amine cation component and an anion component. In some instances, the anion component is bis(fluorosulfonyl)imide (“FSI”).

In some embodiments, the ionic liquid comprises about 100 ppm or less, typically 10 ppm or less, and often 1 ppm or less of said protonated amine compound. In other embodiments, the protonated amine compound that is present in the ionic liquid produced in step (b) is also an ionic liquid.

In an alternative embodiment, the base is added to the quaternary amine compound that is produced in step (a) rather than to the ionic liquid that is produced in step (b) as indicated in step (c).

Another aspect of the invention provides a process for producing a quaternary amine compound comprising:
(a) reacting a tertiary amine compound with an alkylating agent in an aqueous solution under conditions sufficient to produce a crude quaternary amine compound;
(b) adding a base to said crude quaternary amine compound under conditions sufficient to deprotonate a protonated tertiary amine compound that may be present in said crude quaternary amine compound to form said tertiary amine compound, wherein the pKa of a counter acid of said base is higher than the pKa of said protonated tertiary amine compound; and
(c) removing said tertiary amine compound produced in said step (b) to produce said quaternary amine compound.

In some embodiments, the crude quaternary amine compound is isolated prior to said step (b) of adding said base. Still in other embodiments, the process further comprises the step of metathesizing the crude quaternary amine compound to produce an ionic liquid form of said crude quaternary amine compound prior to said step (b) of adding said base.

Yet other aspects of the invention provide an ionic liquid produced from the process disclosed herein.

Some aspects of the invention provide a quaternary amine ionic liquid having less than 1000 ppm, typically less than 100 ppm, and often less than 10 ppm of a tertiary amine ionic liquid. Thus, while quaternary amine ionic liquids of the invention may include a tertiary amine ionic liquid as an impurity, the amount of tertiary amine ionic liquid is significantly less than those obtained from conventional methods.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Reaction of N-methyl-N-propylpyrrolidinium Bromide in water with Hydrogen Bis(fluorosulfonyl)imide followed by treatment with aqueous Sodium Hydroxide: In a 600 mL beaker, aqueous N-methyl-N-propylpyrrolidionium Bromide (Pyr₁₃Br) (50% w/w) (198.8 g with 4000 ppm of protonated N-methylpyrrolidinium (i.e., Pyr1H⁺) and 8000 ppm N-methylpyrrolidine) was stirred while neat hydrogen bis(fluorosulfonyl)imide (HFSI) (81.1 g) was added dropwise from a 250 mL separatory funnel. The temperature of the reaction mixture was kept below 40° C. and was stirred for 20 minutes after the total amount of HFSI was added. The mixture was then transferred into a 250 mL separatory funnel and was shaken for an additional 2 minutes. Once the resultant 2-phase mixture of N-methyl-N-propylpyrrolidinium bis(fluorosulfonyl)imide (i.e., PYR₁₃FSI) and water was settled, the ionic liquid phase was taken and split into two fractions. The first fraction (PYR₁₃FSI, 60.6 g) was mixed with one molar equivalent of sodium hydroxide (8 g) in 30 g of deionized water in a 250 mL beaker and allowed to stir for one hour. After stirring, the 2-phase mixture was again settled and the ionic liquid was separated from the aqueous phase. Both fractions were then separately mixed with equivalent masses of water, shaken in a separatory funnel for 3 minutes, allowed to settle and collected. This washing was repeated six times with an average ionic liquid (i.e., IL) recovery of 96%. Both IL fractions were then mixed with 10% (w/w) activated charcoal for 18 hours and filtered by vacuum. The IL's were then dried at 80° C. for 16 hours under vacuum. The ionic impurities were then measured with ion chromatography with the following results: Hydroxide treated IL: F: 2 ppm Cl: <1 ppm Br: <10 ppm SO4: <50 ppm Pyr_{1H}⁺: <1 ppm; Control (untreated IL): F: 4 ppm Cl: <1 ppm Br: <10 ppm SO4: <50 ppm Pyr_{1H}⁺: 155 ppm.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A method for removing a protonated amine compound from a quaternary amine compound, said method comprising contacting a quaternary amine compound comprising a protonated amine compound with a base under conditions sufficient to remove said protonated amine compound from said quaternary amine compound, wherein the pKa of a counter acid of said base is higher than the pKa of said protonated amine compound.

2. The method of claim 1, wherein said quaternary amine compound is a liquid at a standard condition.

3. The method of claim 1, wherein the pKa of said counter acid of said base is higher than the pKa of said protonated amine compound by at least 1.

4. The method of claim 3, wherein the pKa of said counter acid of said base is higher than the pKa of said protonated amine compound by at least 2.

5. The method of claim 1, wherein said quaternary amine compound is an ionic liquid.

6. The method of claim 1, wherein said base is added to said quaternary amine compound in the presence of an aqueous solution.

7. The method of claim 1, wherein said protonated amine compound is converted to a corresponding amine compound by said base.

8. The method of claim 7, wherein said amine compound is removed from said quaternary amine compound by adsorption.

9. The method of claim 7, wherein said amine compound is removed from said quaternary amine compound as a vapor.

10. The method of claim 1, wherein at least 90% of said protonated amine compound is removed from said quaternary amine compound.

11. A process for producing an ionic liquid comprising a quaternary amine cation, said process comprising:
(a) contacting a tertiary amine compound with an alkylating compound to produce a cationic portion of an ionic liquid, wherein said cationic portion of said ionic liquid is a quaternary amine compound;
(b) admixing said quaternary amine compound with an acid or salt of anionic portion of said ionic liquid to produce said ionic liquid; and
(c) contacting said ionic liquid with a base under conditions sufficient to remove at least a portion of a protonated amine compound that is present in said ionic liquid of said step (b).

12. The process of claim 11, wherein the pKa of a counter acid of said base is higher than the pKa of said protonated amine compound.

13. The process of claim 11, wherein said ionic liquid comprises about 100 ppm or less of said protonated amine compound.

14. The process of claim 11, wherein said protonated amine compound is removed by adsorption.

15. The process of claim 11, wherein at least a portion of said tertiary amine compound is removed as a vapor.

16. A process for producing a quaternary amine compound comprising:
(a) reacting a tertiary amine compound with an alkylating agent in an aqueous solution under conditions sufficient to produce a crude quaternary amine compound;
(b) adding a base to said crude quaternary amine compound under conditions sufficient to deprotonate a protonated tertiary amine compound that may be present in said crude quaternary amine compound to form said tertiary amine compound, wherein the pKa of a counter acid of said base is higher than the pKa of said protonated tertiary amine compound; and
(c) removing said tertiary amine compound produced in said step (b) to produce said quaternary amine compound.

17. The process of claim 16, wherein said crude quaternary amine compound is isolated prior to said step (b) of adding said base.

18. The process of claim 16 further comprising the step of metathesizing said crude quaternary amine compound to produce an ionic liquid form of said crude quaternary amine compound prior to said step (b) of adding said base.

19. The process of claim 16, wherein said tertiary amine compound is removed from said quaternary amine compound by adsorption, as a vapor, solvent extraction or a combination thereof.

20. The process of claim 16, wherein the amount of said tertiary amine compound in said quaternary amine compound is about 100 ppm or less.
